(19) United States
(12) Patent Application Publication
(10) Pub. No.: US 2005/0234317 A1
(43) Pub. Date: Oct. 20, 2005

(54) Low power and personal pulse oximetry systems

(76) Inventor: Massi E. Kiani, Laguna Niguel, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/085,637

(22) Filed: Mar. 21, 2005

Related U.S. Application Data
(60) Provisional application No. 60/554,667, filed on Mar. 19, 2004.
(60) Provisional application No. 60/560,667, filed on Apr. 8, 2004.

(51) Int. Cl. A61B 5/00

(52) U.S. Cl. 600/323

(57) ABSTRACT

Personal pulse oximetry systems and methods are disclosed which provide monitoring, powering, and wireless communications for measurement of an individual's blood oxygen levels in medical, military, or athletic applications. In an embodiment, at least one intensity signals is disabled so as to reduce power consumption.

### REFERENCE TO RELATED APPLICATION

The present application claims priority benefit under 35 U.S.C. §119(e) from U.S. Provisional Application No. 60/554,667, filed Mar. 19, 2004, entitled “Personal Pulse Oximetry Systems and Methods,” and from U.S. Provisional Application No. 60/560,667 filed Apr. 8, 2004, entitled “Low Power Pulse Oximetry,” which are incorporated herein by reference.

### FIELD OF THE INVENTION

The present invention relates to the field of pulse oximetry.

### BACKGROUND OF THE INVENTION

Pulse oximetry is a widely accepted noninvasive procedure for measuring the oxygen saturation level of a person's arterial blood, an indicator of their oxygen supply. Oxygen saturation monitoring is crucial in critical care and surgical applications, where an insufficient blood supply can quickly lead to injury or death. FIG. 1 illustrates a conventional pulse oximetry system 100, which has a sensor 110 and a monitor 150. The sensor 110, which can be attached to an adult's finger or an infant's foot, for example, has both red and infrared LED emitters 112 and a photodiode detector 114. For a finger, the sensor is configured so that the LEDs 112 project light through the fingernail and into the blood vessels and capillaries underneath. The photodiode 114 is positioned at the finger tip opposite the fingernail so as to detect the LED emitted light as it emerges from the finger tissues. A pulse oximetry sensor is described in U.S. Pat. No. 6,088,607 entitled “Low Noise Optical Probe,” which is assigned to Masimo Corporation, Irvine, Calif. and incorporated by reference herein.

Also shown in FIG. 1, the monitor 150 has LED drivers 152, a signal conditioning and digitization front-end 154, a signal processor 156, a display driver 158 and a display 159. The LED drivers 152 alternately activate the red and IR LEDs 112 and the front-end 154 conditions and digitizes the resulting current generated by the photodiode 114, which is indicative of, for example, the intensity of the light detected after attenuation by body tissue. The signal processor 156 inputs the conditioned photodiode signal and determines oxygen saturation based on the differential absorption by arterial blood of the two wavelengths emitted by the LEDs 112. Specifically, a ratio of detected red and infrared intensities is calculated by the signal processor 156, and an arterial oxygen saturation value is determined based on the ratio obtained. The display driver 158 and associated display 159 indicate a patient's oxygen saturation, heart rate, plethysmographic waveform, or the like. Pulse oximetry signal processing is described in U.S. Pat. Nos. 5,782,757, 6,650,917 and 6,699,194, which are assigned to Masimo Corporation, Irvine, Calif. and incorporated by reference herein.

Probes, such as the sensor 110, however, are dependent on an external pulse oximeter, such as the monitor 150, to function. The signal detected is sent, usually via cable 160, to an external pulse oximeter that provides power to the sensor 110 and analysis of the probe output by the monitor 150. The output, once analyzed, is displayed, recorded or monitored by the monitor 150, which often provides alarms, outputs compatible with wider patient monitoring networks using various communication protocols, or the like.

External pulse oximeters often range large in size, such as from approximately the size of a laptop computer, to that of a desktop computer, to multiparameter systems. Circuit boards for use in external pulse oximeters are also available, but suffer from similar drawbacks, i.e. these board level products cannot be used on their own without a host device providing regulated power, serial communication, monitoring and alarm processing, and information display.

In conventional systems, the sensor 110 is also physically tethered to the monitor 150. Such a tether has several drawbacks for medical patients during care, and prevents the use of pulse oximetry probes in other arenas where continual monitoring of an individual's vital statistics are warranted. For example, in military applications, physical therapy, or sports applications, the tethering of a soldier, patient or athlete to an external pulse oximeter is impractical and could be dangerous. Such tethering can also render other consumer applications of pulse oximetry more difficult.

Furthermore, external pulse oximeters themselves are often large in size, expensive, encumbered by power cords, and restrained by communication cables thus often not permitting their use as for many medical, military, sports, or consumer applications. As a result, the traditional combination of a cable tether, pulse oximetry probe, and a non-portable external pulse oximeter greatly limits the use and applications of pulse oximetry, especially outside the medical field.

Embodiments of the present invention seek to overcome some or all of these and other problems.

### SUMMARY OF THE INVENTION

One aspect of low power pulse oximetry provides at least first and second intensity signals generated by the detection of light having at least first and second wavelengths after absorption by constituents of pulsatile blood flowing within a fleshy medium. The intensity signals are processed so as to provide a physiological measurement. At least one of the intensity signals is then disabled so as to reduce power consumption. The method may further comprise the step of establishing a baseline measurement responsive to another one of the intensity signals. A subsequent measurement responsive to that intensity signal is provided. The subsequent measurement is compared to the baseline measurement and the disabled intensity signal is re-enabled in response. In one embodiment, the disabling step comprises the substep of deactivating at least one emitter of a sensor adapted to attach to fleshy media. In a particular embodiment, drive current to at least one emitter is disabled.

Another aspect of low power pulse oximetry provides a first intensity signal generated by the detection of light having a first wavelength after absorption by constituents of pulsatile blood flowing within a fleshy medium. A second intensity signal is enabled in response to the first intensity signal, where the second intensity signal is generated by the detection of light having a second wavelength after absorption by constituents of pulsatile blood flowing within a fleshy medium. The first and second intensity signals are processed so as to measure a physiological parameter. The method may further comprise the step of establishing a baseline measurement responsive to the first intensity signal. A subsequent measurement responsive to the first intensity signal is provided. The subsequent measurement is compared to the baseline measurement so as to determine whether to enable the second intensity signal. In one embodiment, the enabling step comprises the substep of activating at least one emitter of a sensor adapted to attach to fleshy media. In a particular embodiment, the activating substep comprises the substep of enabling drive current to the emitter or emitters.

A further aspect of low power pulse oximetry establishes a baseline measurement responsive to at least one of first and second intensity signals generated by the detection of light having at least first and second wavelengths after absorption by constituents of pulsatile blood flowing within a fleshy medium. A subsequent measurement responsive to at least one of the intensity signals is provided. The subsequent measurement is compared to the baseline measurement. A signal processing technique relating to at least one of the intensity signals is intermittently foregone so as to reduce power consumption. The signal processing technique may be restarted in response to the comparing step. In one embodiment, the signal processing technique is foregone by disabling drive current to a sensor emitter, and the signal processing technique is restarted by enabling drive current to the emitter.

Yet another aspect of low power pulse oximetry comprises a sensor having first and second emitters adapted to transmit light of first and second wavelengths into a fleshy medium. A light sensitive detector is adapted to generate first and second intensity signals by detecting the light after absorption by constituents of pulsatile blood flowing within the fleshy medium. A monitor is configured to accept the intensity signals, generate digitized signals from the intensity signals and compute at least one physiological parameter responsive to magnitudes of the digitized signals. In one embodiment, the first emitter is disabled during a first time period. In another embodiment, the second intensity signal is monitored during this first time period. If the second intensity signal changes by more than a predetermined amount, the first emitter can be re-enabled.

Aspects of the disclosure also include a personal pulse oximeter (“personal pulse oximeter”) which operates as a portable/wearable pulse oximeter that permits both wired and wireless communication between the personal pulse oximeter and medical, military or general communications networks, without requiring a cable tether to a pulse oximetry probe.

In an embodiment, the personal pulse oximeter does not require a cable tether to a sensor or pulse oximetry probe, and can operate as a self-powered, fully functional pulse oximeter while providing portability and/or wearability by an individual, and advanced communication and networking technology for compatibility with medical, military or general communications networks. In addition, such a personal pulse oximeter can provide easy exchange, reduced repair and replacement costs, personal identification and authentication for users, combinations of the same or the like, even beyond the medical realm.

In an embodiment, the personal pulse oximeter includes a wireless communications link to provide wireless communications between the personal pulse oximeter and external devices such as, for example, an external pulse oximeter. In an embodiment, a processor computes a pulse oximetry profile based on information communicated from a pulse oximetry probe via a communications link. In an embodiment, a display shows information from the processor or received via a communications link. An input device can be used for sending information to the processor or to an external device via a communications link.

In an embodiment, the personal pulse oximeter includes an input module, an antenna to provide communications between the oximeter and external devices through at least one communications protocol, and one or more ports to provide communications between the oximeter and external devices through at least one communications protocol. A pulse oximetry probe communicates with the foregoing personal pulse oximeter through at least one of the port and the antenna. In an embodiment, the personal pulse oximeter includes an alarm.

In an embodiment, an wireless adapter is provided for use with a pulse oximeter. The wireless adapter includes a sensor connector configured to couple the wireless cable connector to a pulse oximetry sensor. A transceiver and antenna provide wireless communications between the wireless adapter and the pulse oximeter. In an embodiment, a personal pulse oximeter includes a processor for controlling data flow in the wireless adapter. In an embodiment, the wireless adapter includes a display to show signal of status and/or battery status for the wireless adapter.

For purposes of summarizing the invention, certain aspects, advantages and novel features of the invention have been described herein. Of course, it is to be understood that not necessarily all such aspects, advantages or features will be embodied in any particular embodiment of the invention.

### BRIEF DESCRIPTION OF THE DRAWINGS

FIG. 1 is a block diagram of a conventional pulse oximeter sensor and monitor;

FIG. 2 is a flowchart of a low power pulse oximetry process; and

FIG. 3 is a graph of emitter drive current versus time for a low power pulse oximetry process.

FIG. 4 is a top view of a simplified embodiment of a personal pulse oximeter module.

FIG. 5 is a top view of a simplified embodiment of a wearable personal pulse oximeter module.

FIG. 6 is a top view of a simplified embodiment of an wireless adapter for a pulse oximetry probe used with a pulse oximeter.

FIG. 7 is a functional chart of a simplified embodiment of a personal pulse oximetry system.

FIG. 8 is a functional chart of a simplified embodiment of an wireless adapter for use with a personal pulse oximetry system.

FIG. 9 is a functional chart of a simplified embodiment of a personal pulse oximetry system.

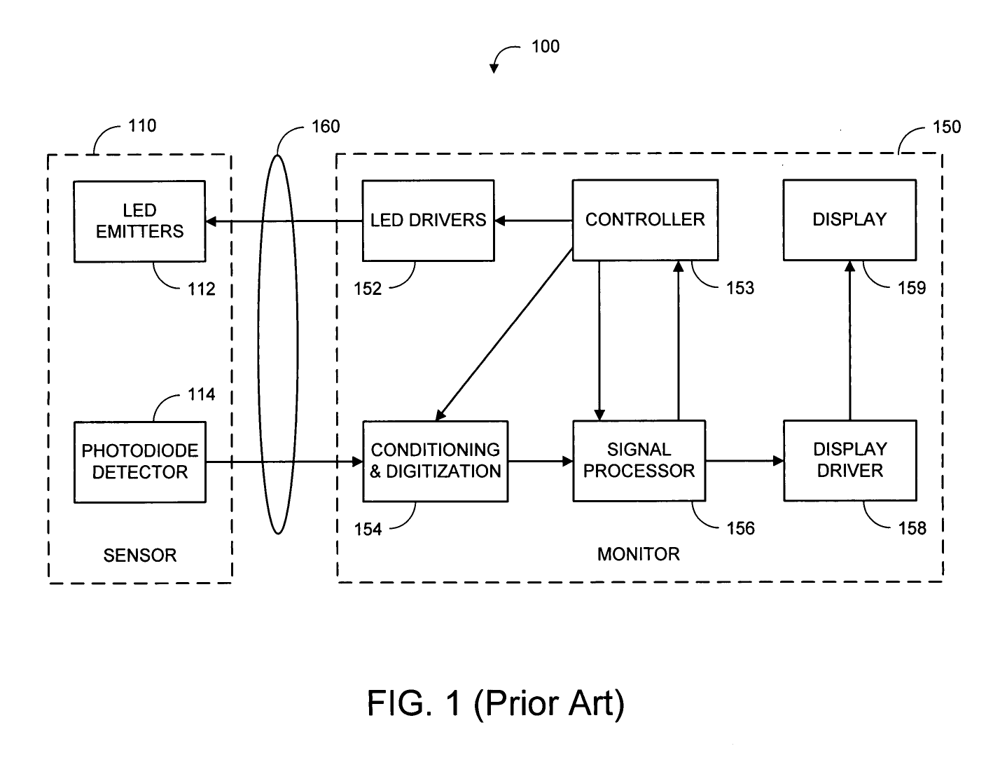

### DETAILED DESCRIPTION OF THE PREFERRED EMBODIMENT

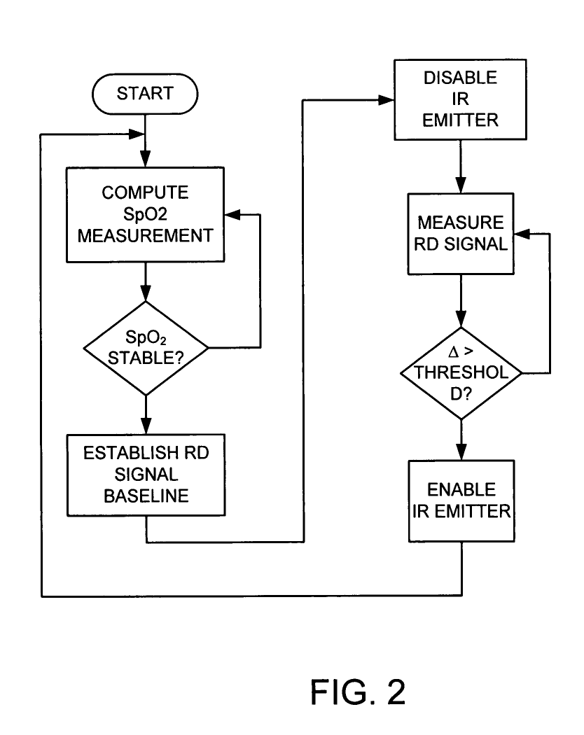

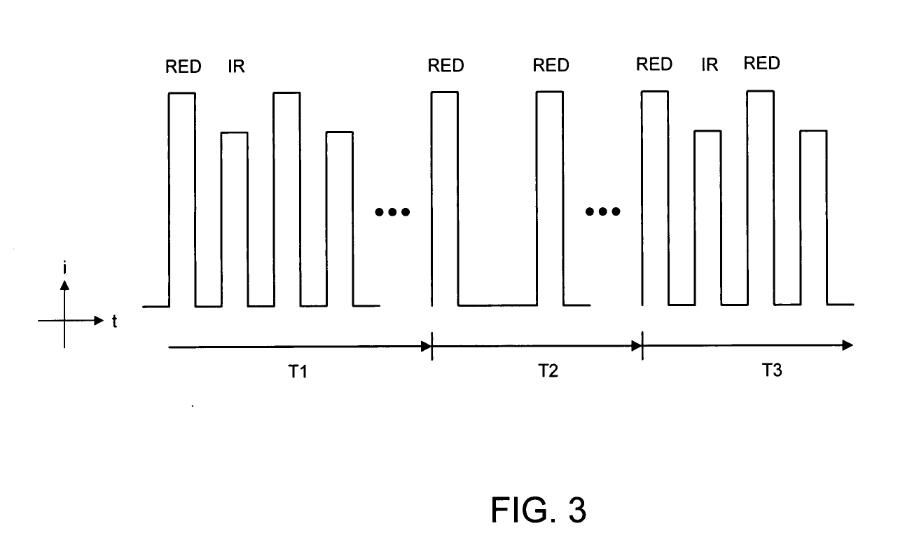

FIGS. 2-3 illustrate an exemplary low power pulse oximetry process. During a first time period T1 (FIG. 3), both RD (red) and IR (infrared) emitters are enabled and SpO₂ measurements are computed and displayed. If the SpO₂ measurements are stable, i.e. the values do not change more than a predetermined amount during a predetermined time interval, then a RD signal baseline is established. The baseline may be, for example, an average of the AC component of the RD signal. The IR emitter is then disabled during a second time period T2 (FIG. 3). In an embodiment, the RD signal is periodically measured and compared to the baseline value. If the absolute difference (Δ) is greater than a predetermined threshold, then the IR emitter is re-enabled. During this third time period T3 (FIG. 3), SpO₂ measurements are once again computed. Although a low power pulse oximetry process is described above with respect to enabling and disabling an IR emitter and periodically measuring a RD emitter, the process is also applicable with respect to enabling and disabling a RD emitter and periodically measuring an IR emitter.

In general terms, once a baseline measurement is established, regardless of the particular variables used for the baseline, the signal processing may determine to reduce one or more signal processing techniques so as to reduce power consumption or the like. In one embodiment, the signal processing may determine to reduce the number of LEDs used, such as, for example, eliminating one or more LED drive signals. In another embodiment, the signal processing may determine to forego one or more processing techniques used to either process the intensity data and/or compute SpO₂. One the signal processing determines that a threshold difference has been met between the baseline and current data, the signal processing can effectively restart or enable one or more of the processing techniques previously foregone.

Low power pulse oximetry has been disclosed in detail in connection with various embodiments. These embodiments are disclosed by way of examples only and are not to limit the scope of the claims that follow. One of ordinary skill in art will appreciate many variations and modifications from the disclosure herein.

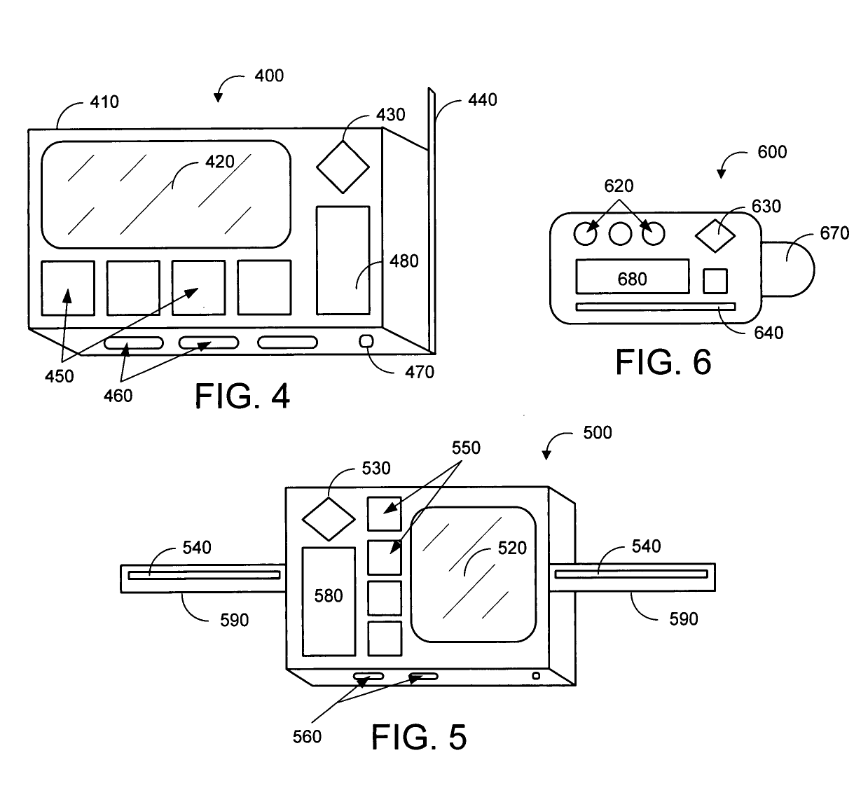

FIG. 4 shows one embodiment of a portable oximeter module 400. The module 400 includes a case 410, a display 420, an audio device 430, an antenna 440, one or more input buttons 450, one or more power sources 480 (e.g., batteries, fuel cells, etc.) and one or more ports 460. A pulse oximeter probe is attached to the patient and communicates with the module 400 (directly, wirelessly, or the like). A skilled artisan will recognize from the disclosure herein a wide number of known or developed technologies and/or protocols for providing robust wireless communications over any FCC-acceptable frequency range. Moreover, such communication may be automatically detected or otherwise menu selectable by the module 100. For example, the communication may include software designable wireless systems, where software detects and/or selects which wireless communication standard or protocol may be employed to govern current communication. Such systems may select protocols based on interference on alternative selections, power consumption issues, detected protocols, security issues such as encryption, hardware limitations, model numbers, combinations of the same, or the like.

The module 400 drives one or more light emitting diodes in the probe to generate light that propagates through the tissue of a patient. A detector on the probe detects light that propagates through the tissue and provides a data signal to the module 400. In an embodiment, the module 400 analyzes the data signal to determine one or more physiological parameters of the patient (e.g., pulserate, blood oxygen saturation, etc.). However, an artisan will also recognize from the disclosure herein that in order to reduce power, size, and/or cost, the module 400 may advantageously provide the data signal (or data corresponding to the data signal) to an external pulse oximeter unit that determines one or more physiological parameters, provide pre-processing of the data before providing the data to the external pulse oximeter, or the like. In an embodiment, the external pulse oximeter may advantageously send data back to the module 400 to be displayed on the display 420, trigger alarms or other audio or video signaling, or the like.

FIG. 5 shows one embodiment of a wearable oximeter module 500. The wearable module 500 includes an antenna 540 for wireless communication, and one or more connectors 560 for connecting to a pulse oximeter probe. Optionally, the oximeter 500 includes a display 520, an audio device 530, one or more input buttons 550, one or more power sources 580 (e.g., batteries, fuel cells, etc.), and a binding 590 for attaching the module 500 to a patient. The binding can include, for example, a watch strap, a belt, a headband, clothing, or the like.

A pulse oximeter probe is attached to the patient and communicates with the module 500 (directly, wirelessly, or the like). Similar to the foregoing, a skilled artisan will recognize from the disclosure herein a wide number of technologies and/or protocols for providing robust wireless communications and/or software. The module 500 drives one or more light emitting diodes in the probe to generate light. A detector on the probe detects light after attenuation by body tissue of the patient and provides a data signal to the module 500. In an embodiment, the module 500 includes a pulse oximeter processor signal processing system that analyzes the data signal to determine one or more physiological parameters of the patient (e.g., pulserate, blood oxygen saturation, etc.).

However, an artisan will also recognize from the disclosure herein that in order to reduce power, size, and/or cost, the module 500 may advantageously provide the data signal (or data corresponding to the data signal) to an external pulse oximeter unit that determines one or more physiological parameters, provide pre-processing of the data before providing the data to the external pulse oximeter, or the like. In an embodiment, the external pulse oximeter may advantageously send data back to the module 500 to be displayed on the display 520, trigger alarms or other audio or video signaling, or the like

In an embodiment, the oximeter modules 400, 500 provide low power consumption, wireless capability, patient location capability, and support for additional features and functions through one or more interface ports. The oximeter modules 400, 500 reduce or eliminate the reliance on a host device, reduce power consumption to levels acceptable for ambulatory battery-powered devices, and support peripheral devices and features via one or more interface port (wireless, location/tracking, trend storage and retrieval, etc.) as desired.

In an embodiment, the oximeter modules 400, 500 communicate physiologic data and provide location tracking (e.g., sensor data, pulse rates, oxygen saturation, etc.) using telemetry networks, such as WMTS compatible networks, to communicate with external monitors or monitoring. Wireless Medical Telemetry Service (WMTS) has been approved by the FCC for monitoring patient physiological parameters over a distance via radio-frequency (RF) communications between, for example, a transmitter worn by the patient and a central monitoring station. It appears that the FCC will set aside the frequencies of: 608 to 614 MHz, 1395 to 1400 MHz, and 1429 to 1432 MHz for primary or co-primary use by wireless medical telemetry users. As disclosed in the foregoing, wireless communication includes the advantage of allowing patient movement without tethering the patient to a bedside monitor with a hard-wired connection. As will be recognized by an artisan from the disclosure herein, a wide number of wireless communication protocols and frequencies could be used for wireless communication, location tracking, and the like.

Additionally, the modules 400, 500 can provide patient (or device) tracking systems using GPS or other location systems, allowing clinicians to locate the patient (or device) within, for example, an emergency care environment, a general medical care or monitoring environment, a military environment, or the like. Moreover, such tracking provides ready solutions in the event the monitor is misplaced or if the patient requires medical intervention.

FIG. 6 shows a wireless adapter 600 capable supporting wireless communication between, for example, a convention pulse oximeter or the monitors 400, 500, and a sensor or probe. In an embodiment, the wireless adapter 600 includes a connector 670 for connecting to a pulse oximetry probe, one or more power sources 680 (e.g., batteries, fuel cells, etc.), a transceiver (not shown), and an antenna 640. The wireless adapter 600 optionally includes display elements 620, a display, an audio input/output device 630, one or more communication ports, or the like. In an embodiment, the connector 670 is mechanically adapted to connect to any number of conventional oximetry sensors or probes, including disposable, reusable, or combination sensors. For example, in an embodiment, the connector 670 may comprise mechanical mating portions similar to those disclosed in U.S. Pat. Nos. 5,645,440 and D393,830 which are assigned to Masimo Corporation, Irvine, Calif. and incorporated by reference herein.

In the embodiment of FIG. 6, the probe is attached to the patient and provided to the wireless adapter 600. The wireless adapter 600 receives data from a pulse oximeter to drive one or more light emitting diodes in the probe to generate light that propagates through tissue of the patient. In an embodiment, the data comprises emitter drive signal(s). In other embodiments, the data comprises instructions sufficient for the wireless adapter to generate emitter drive signal(s). A detector on the probe detects light that propagates through the patient and provides a data signal to the wireless adapter 600. The wireless adapter 600 provides the data signal (or data corresponding to the data signal) to the external pulse oximeter, which uses the data to determine one or more physiological parameters of the patient (e.g., pulserate, blood oxygen saturation, etc.).

In an embodiment, the wireless adapter 600 pre-processes the data before providing the sensor data to the pulse oximeter system. In an embodiment, the pulse oximeter system sends commands to the wireless adapter 600 to control the operation of the pulse oximeter probe. In an embodiment, the pulse oximeter system sends data back to the wireless adapter 600 to trigger alarms or other audio signaling on the audio device 630.

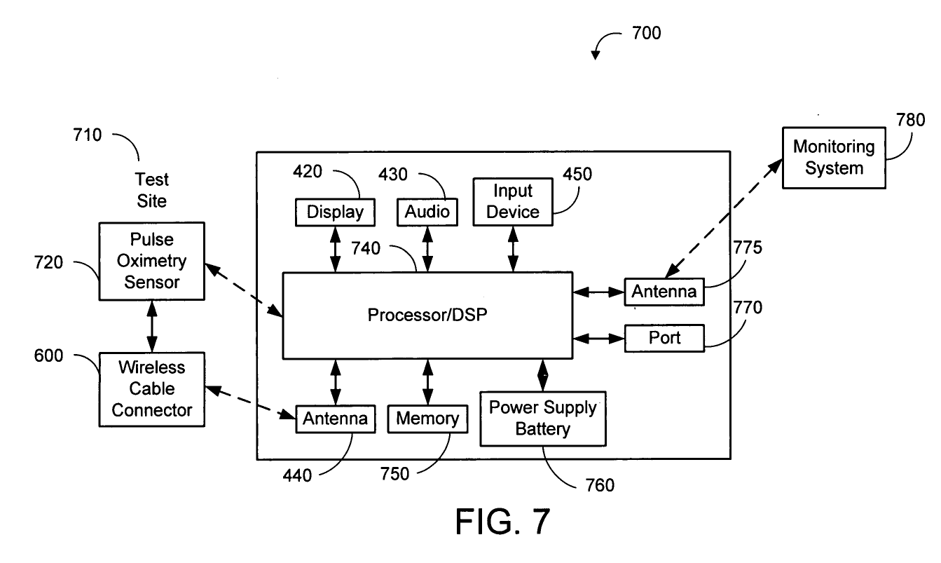

FIG. 7 is a block diagram 700 showing one embodiment of a personal pulse oximetry system. In the diagram 700, a test site 710 (on the patient) is irradiated with light by a pulse oximeter probe 720. The probe 720 detects the lights after attenuation by the body tissue at the test site 710, and provides a signal representative of the detected light to a wireless adapter 600. The wireless adapter 600 communicates with antenna 440 in the pulse oximeter module 400. The antenna 440 communicates with a processor 740. In an embodiment, the processor 740 includes frequency processing to demodulate the communication signal received by the antenna 440 and to provide modulated communication signals to the antenna 440.

In an embodiment, the processor 740 receives the data from the wireless adapter 600 and performs signal processing on the data. For example, the processor 740 may determine one or more physiological parameters, may preprocess the data, may forward raw data, processed data, or determined values for the monitored parameters to an external monitoring system 780 through an antenna 780, combinations of the same, or the like. In an embodiment, processed data, and/or physiological parameters from the processor 740 are modulated onto a radio-frequency communication signal and provided to the antenna 775. An artisan will recognize from the disclosure herein that the antenna 775 is optional and that in another embodiment, the processor 740 can communicate directly with the external monitoring system 740 or through the antenna 440. In an embodiment, processed data, and/or physiological parameters from the processor 740 are provided to a communication port 770. In an embodiment, the processor 740 also provides data (e.g., pulserate, status information, blood oxygen saturation, etc.) to the display 420. Power for the module 400 is provided by a power source 760 (e.g., a battery, a fuel cell, a power supply, etc.).

Although disclosed with reference to FIG. 7, an artisan will recognize from the disclosure herein a wide variety of personal oximeters that accept communication of wireless or wired sensors.

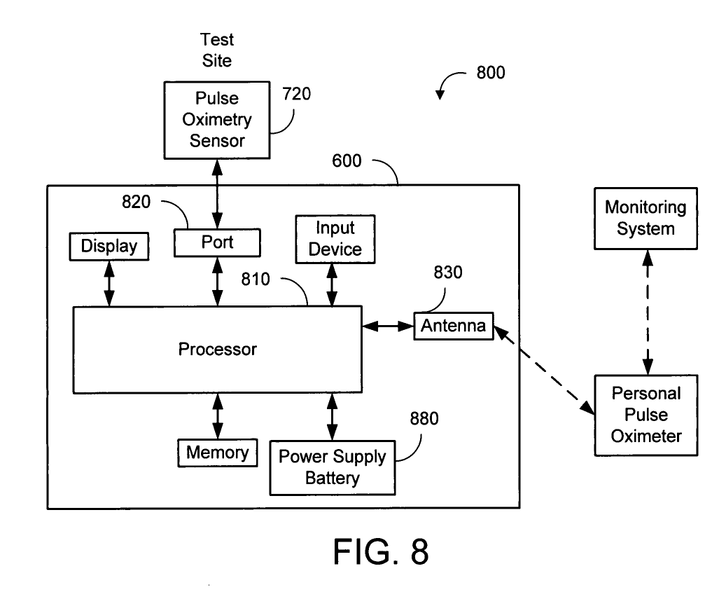

FIG. 8 is a block diagram 800 showing one embodiment of a wireless adapter 600. In the diagram 800, the pulse oximeter probe 720 communicates with a processor 810 through a port 820. The processor 810 generates signals to control one or more light sources in the sensor 720. The processor 810 receives sensor data from an optical detector in the probe 720. The processor 810 performs signal processing on the sensor data, such as, for example, modulating the sensor data to a radio-frequency communication signal and providing the same to the antenna 830 for transmission to antenna 440 of the oximeter of FIG. 7. Power for the wireless adapter 600 can be provided by a power source 880 (e.g., a battery, a fuel cell, a power supply, etc.), although an artisan will recognize other powering solutions, including locally carried power supplies such as, for example, other monitoring devices or other equipment.

Although disclosed with reference to FIG. 8, an artisan will recognize from the disclosure herein a wide variety of wireless adapters that communicate detected data from a test site to a monitoring device capable of determining values of desired monitored parameters.

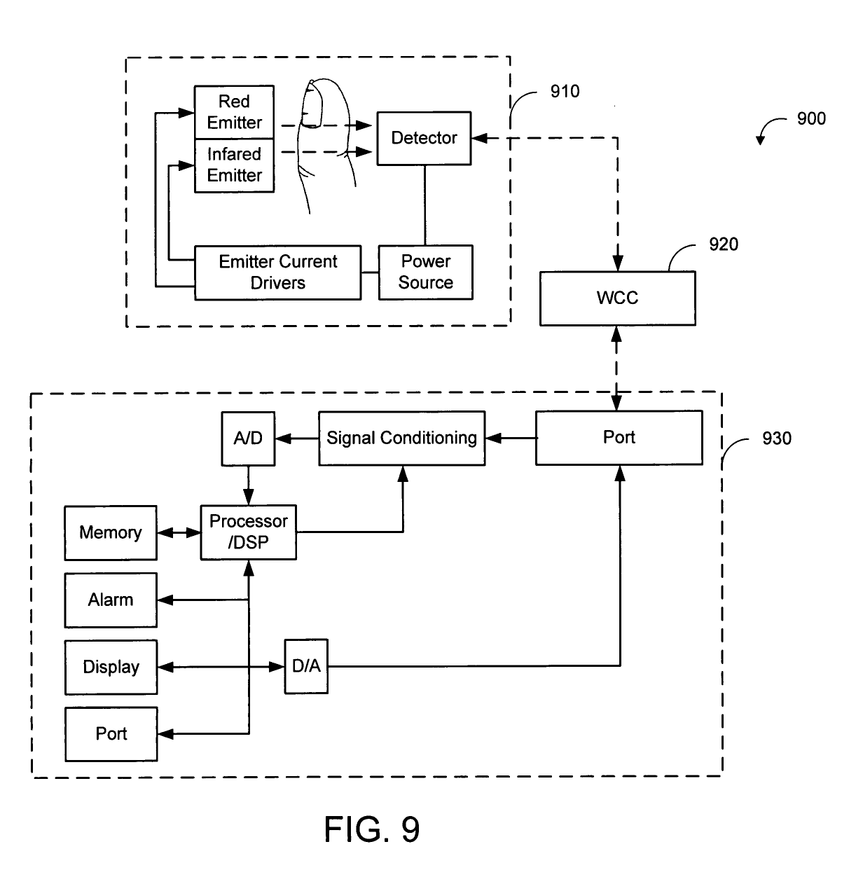

FIG. 9 illustrates a block diagram of yet another embodiment of a wireless pulse oximetry system including a sensor 910, a wireless adapter 920, such as, for example, the wireless adapter 600, and a personal pulse oximeter 930. As shown in FIG. 9, the sensor 910 drives the emitters to emit light detectable by a detector after attenuation by body tissue. The detector communicates the detected signal to the oximeter 930 through the adapter 920. The oximeter 930 determines one or more characteristics of the body tissue.

A skilled artisan will recognize from the disclosure herein a wide number of other embodiments, including but not limited to, changes in the shape and layout of the personal pulse oximeter and its components, alternative communications protocols, alternative wireless and wired cable connector designs, merging of the wearable personal pulse oximeter and pulse oximetry probe in one device, and use of the wearable personal pulse oximeter in combination with apparel, jewelry, timepieces, personal digital assistants, and the like.

In addition to the foregoing, one or more of the embodiments disclosed here can implement a communication protocol capable of using the body's chemistry to propagate information between sensor and signal processing devices. For example, signals may be pre-processed or not, at the sensor, and then transmitted as a low energy signal through the skin. The personal pulse oximeter in this embodiment receives the signal propagated through body tissue and performs appropriate processing in order to determine one or more physiological characteristics of the wearer. In an embodiment, the signal propagated through body tissue may be encoded to increase the ability to be detectable, e.g. propagated as encoded digital or binary information.

The foregoing use of the body tissue to as a signal transmission medium provides for wireless signal transmission that is more difficult to detect by other devices. Moreover, such transmission provides for decreased cross-talk between wearers of wireless systems. These and other advantages are especially helpful in many applications, including military or other stealth environments.

Other combinations or modifications will also be recognized by a skilled artisan from the disclosure herein. Moreover, the described embodiments and examples are to be considered in all respects, only as illustrative and not restrictive. The scope of the invention therefore is indicated by the appended claims rather than by the foregoing description.

Additionally, all publications, patents, and patent applications mentioned in this specification are herein incorporated by reference to the same extent as if each individual publication, patent, or patent application was specifically and individually indicated to be incorporated by reference.

1. A method of reducing power used in a pulse oximetry system, the method comprising the steps of:
receiving intensity signals generated by the detection of light attenuated by a fleshy medium;
processing said intensity signals so as to provide a physiological measurement; and
disabling at least one of said intensity signals so as to reduce power consumption.

2. The method according to claim 1 further comprising the step of:
establishing a baseline measurement responsive to at least another one of said intensity signals;
providing a subsequent measurement responsive to said at least another one of said intensity signals;
comparing said subsequent measurement to said baseline measurement; and
re-enabling said at least one of said intensity signals in response to said comparing step.

3. The method according to claim 1 wherein said disabling step comprises the substep of deactivating at least one emitter of a sensor adapted to attach to fleshy media.

4. The method according to claim 3 wherein said deactivating substep comprises the substep of disabling drive current to said at least one emitter.

5. A low power pulse oximetry method comprising the steps of:
establishing a baseline measurement responsive to at least one of first and second intensity signals generated by the detection of light having at least first and second wavelengths after absorption by constituents of pulsatile blood flowing within a fleshy medium;
providing a subsequent measurement responsive to at least one of said intensity signals;
comparing said subsequent measurement to said baseline measurement; and
based on said comparing, foregoing a signal processing technique relating to at least one of said intensity signals so as to reduce power consumption.

6. The low power pulse oximetry method according to claim 5, further comprising restarting said signal processing technique in response to said comparing step.

7. The low power pulse oximetry method according to claim 6 wherein:
said foregoing step comprises the substep of disabling drive current to a sensor emitter; and
said restarting step comprises the substep of enabling drive current to said emitter.

8. A monitoring system comprising:
a personal pulse oximeter configured to process one or more intensity signals indicative of one or more physiological parameters of a monitored patient;
a sensor configured to output the one or more intensity signals;
a wireless adapter configured to control communication between the personal pulse oximeter and the sensor; and
an external patient monitoring system capable of communicating with the personal pulse oximeter.

9. The monitoring system of claim 8, wherein the communication between the external patient monitoring system and the personal pulse oximeter includes location tracking data sufficient for the external patient monitoring system to track a location of the personal pulse oximeter.

10. The monitoring system of claim 8, wherein the external patient monitoring system includes software capable of determining a wireless communication protocol being used by the personal pulse oximeter and configures the external monitoring system to receive the data according to the protocol.

11. The monitoring system of claim 8, wherein the wireless adapter communicates signals through body tissue.
